(19) United States
(12) Patent Application Publication
(10) Pub. No.: US 2026/0263520 A1
(43) Pub. Date: Sep. 10, 2026

(54) BINDING MOLCEULE AGAINST P95 HER2 VARIANTS

(71) Applicant: OSLO UNIVERSITETSSYKEHUS HF, Oslo (NO)

(72) Inventors: Jon Amund KYTE, Hosle (NO); S. Esmaeil DORRAJI, Oslo (NO)

(21) Appl. No.: 19/474,161

(22) PCT Filed: Apr. 26, 2024
(86) PCT No.: PCT/EP2024/061560

§ 371 Date: Oct. 9, 2025

(30) Foreign Application Priority Data
Apr. 27, 2023 (GB) 2306268.0

(51) Int. Cl. A61K 35/17; A61K 40/31; A61K 40/42; C07K 14/705; C07K 16/32

(52) U.S. Cl. CPC A61K 35/17; A61K 40/31; A61K 40/4205; C07K 14/7051; C07K 14/70517; C07K 14/70578; C07K 16/32; A61K 2239/13; A61K 2239/17; A61K 2239/21; C07K 2317/53; C07K 2317/565; C07K 2317/622; C07K 2317/92; C07K 2319/03

(57) ABSTRACT

The invention relates to binding molecules which specifically bind p95HER2 comprising the amino acid sequence set forth in SEQ ID NO: 1, comprising a light chain variable domain (VL) and a heavy chain variable domain (VH) which together form an antigen binding unit, wherein: (i) the VL comprises three complementarity determining regions (CDRs) which respectively comprise the amino acid sequences SEQ ID NOs: 2, 3 and 4; and the VH comprises three CDRs which respectively comprise the amino acid sequences SEQ ID NOs: 5, 6 and 7; or (ii) the VL comprises three CDRs which respectively comprise the amino acid sequences SEQ ID NOs: 8, 3 and 9; and ori the VL comprises three CDRs which respectively comprise the amino acid sequences SEQ ID NOs: 5, 6 and 7. The binding molecules represent novel targeting units which have utility in cancer therapy and diagnostics and may be provided in the form of chimeric antigen receptors (CARs).

### TECHNICAL FIELD OF THE INVENTION

The invention is related to the field of cancer therapy and diagnostics. In particular, it relates to novel targeting units and binding molecules, specifically binding proteins or constructs, or chimeric antigen receptors (CARs), comprising them, nucleic acids encoding the targeting units, nucleic acids encoding the binding proteins or CARs, immune cells expressing the binding proteins or CARs and their utility for treatment of cancer.

### BACKGROUND

Many HER2+ breast cancers express isoforms of HER2 with truncated carboxy-terminal fragments (CTF), collectively known as p95HER2.

Some antigen binding units for binding to p95HER2 are known, but not all of them are suitable for implementation into CARs. This is illustrated by the failures described in the Research Disclosure RD667070 published 17 Oct. 2019.

In order to achieve a therapeutic CAR-T cell (CAR-T), the cell needs to express the CAR in a sufficient amount in the cell membrane, and the antigen binding unit has to convey sufficient affinity and specificity for the target antigen. It can be expected that only a fraction of CAR-T cells with in vitro activity will successfully migrate to tumor metastases in vivo and/or infiltrate the hostile tumor microenvironment of a solid tumor. Furthermore, the CAR-T cells will likely need to sustain their activity over time in order to provide a therapeutic effect in vivo. It is therefore not trivial, but very desirable to obtain novel CARs able to provide a therapeutic effect for solid tumors in vivo when the CARs are expressed in the cell membrane of immune cells.

Antigen binding units may also have therapeutic utility in other formats, including as antibodies and antibody-based constructs, e.g. conjugates, including with drugs, and in bi- or tri- or other multi-specific formats where the antigen binding unit is combined with other binding units having different specificity.

A novel anti-HER2 antibody capable of binding specifically to p95HER2 is described in PCT/EP2022/079110, identified herein as the Oslo-2 antibody. This antibody has been shown to be suitable for implementation as an scFv into a CAR and to retain sufficient affinity and specificity for p95HER2 in this format. In the context of developing the Oslo-2 antibody for clinical use, variants of this antibody have been developed and investigated, some of which are the subject of the invention herein.

### SUMMARY

Based on structural modelling of the Oslo-2 antibody:p95HER2 complex and in silico mutational analysis studies, various mutations in the variable domains of the antibody were generated and studied in different combinations. This has led to the identification of a number of mutants, or antibody variants, of Oslo-2, comprising mutations in the CDRs. Based on further analysis of the mutants and optimization for expression in mammalian cells, two particular mutants have been selected and successfully expressed.

Accordingly, provided herein are binding molecules comprising a novel antigen binding unit based on said variant variable domain sequences. Said antigen binding unit, and binding molecules comprising it, are able to specifically bind to cells expressing the hyperactive 611-CTF isoform of p95HER2 under physiological conditions. The antigen binding units herein display little or no binding to full length HER2 under physiological conditions and they display little or no cross-reactivity to healthy tissue. An antibody comprising the novel antigen binding unit displayed a similar binding capacity as determined by ELISA to an antigen derived from p95HER2 in a similar order or range to that of the parental Oslo-2 antibody. In particular, the novel variant antibodies herein comprise mutations in the light chain CDRs, and more particularly in LCDR1, or in LCDR1 and LCDR3.

In a first aspect, we provide a binding molecule which specifically binds p95HER2 comprising the amino acid sequence set forth in SEQ ID NO: 1, comprising a VL and a VH which together form an antigen binding unit, wherein
(i) the VL comprises three complementarity determining regions (CDRs): LCDR1, LCDR2 and LCDR3 which respectively comprise the amino acid sequences SEQ ID NOs: 2, 3 and 4; and
the VH comprises three CDRs: HCDR1, HCDR2 and HCDR3 which respectively comprise the amino acid sequences SEQ ID NOs: 5, 6 and 7; or
(ii) the VL comprises three complementarity determining regions (CDRs): LCDR1, LCDR2 and LCDR3, which respectively comprise the amino acid sequences SEQ ID NOs: 8, 3 and 9; and
the VH comprises three CDRs: HCDR1, HCDR2 and HCDR3, which respectively comprise the amino acid sequences SEQ ID NOs: 5, 6 and 7.

In variant (i) (mutant 4 in the Examples below), the heavy chain CDRs are unchanged over the parental antibody from which the binding molecule is derived, and the VL comprises the mutations S31W in CDR1 and H107W in CDR3.

In variant (ii) (mutant 5 in the Examples below), the heavy chain CDRs are unchanged over the parental antibody from which the binding molecule is derived, and the VL comprises the mutation S31M in CDR1.

In an embodiment, said binding molecule may comprise a VH comprising the amino acid sequence set forth in SEQ ID NO: 10 or a sequence with at least 80, 85 or 90% identity thereto, and a VL comprising the amino acid sequence set forth in SEQ ID NO: 11 or a sequence with at least 80, 85 or 90% identity thereto.

In another embodiment, said binding molecule may comprise a VH comprising the amino acid sequence set forth in SEQ ID NO: 10 or a sequence with at least 80, 85 or 90% identity thereto, and a VL comprising the amino acid sequence set forth in SEQ ID NO: 12 or a sequence with at least 80, 85 or 90% identity thereto.

In an embodiment, the binding molecule is in the format of an antibody, which may be a full-length antibody, or a fragment thereof, or an antibody derivative, including a single-chain antibody.

Said antigen binding unit may be a scFv.

In a second aspect, we provide a Chimeric Antigen Receptor (CAR) comprising an antigen binding unit according to the first aspect. The CAR may comprise a human CD8α hinge.

The CAR may comprise, from N-terminal to C-terminal, a human CD8α hinge, a human CD8α transmembrane domain, a human 4-1BB costimulatory domain and a human CD3ζ signaling domain.

In a third aspect, we provide a nucleic acid encoding a binding molecule according to the first aspect or a CAR according to the second aspect.

In a fourth aspect, we provide a vector comprising the nucleic acid of the third aspect.

In a fifth aspect, we provide an immune cell, and in particular a cytotoxic immune cell, expressing a CAR according to the second aspect in its cell membrane.

In a sixth aspect, we provide a pharmaceutical composition comprising a binding molecule according to the first aspect, a nucleic acid according to the third aspect, a vector according to the fourth aspect or an immune cell, particularly a cytotoxic immune cell, according to the fifth aspect.

In a seventh aspect, we provide a method of treatment of cancer in a subject, particularly a human subject, or in other words in a human patient, comprising the step of administering the immune cell, particularly the cytotoxic immune cell, of the fifth aspect or the pharmaceutical composition of the sixth aspect.

In an eighth aspect, we provide a method of treatment of cancer in a subject, particularly in a human patient, comprising the steps:
(a) obtaining a sample comprising cancer cells from the subject, e.g. patient;
(b) analysing whether the cancer cells express p95HER2 by contacting the cells ex vivo with a binding molecule according to the first aspect further comprising a moiety suitable for detection; and
(c) administering a therapy, particularly an approved therapy, e.g. chemotherapy, to the subject, e.g. patient if the cancer cells are p95HER2 positive.

In a ninth aspect, we provide a method of diagnosing cancer comprising the steps:
(a) obtaining a sample comprising cells from a subject, e.g. a human patient;
(b) analysing whether the cells express p95HER2 by contacting the cells ex vivo with a binding molecule according to the first aspect, wherein the binding molecule comprises a moiety suitable for detection; and
(c) diagnosing the subject, e.g. patient, with cancer if the cells express p95HER2.

In a tenth aspect, we provide a binding molecule according to the first aspect, a CAR according to the second aspect, an immune cell, e.g. a cytotoxic immune cell, according to the fifth aspect or a pharmaceutical composition according to the sixth aspect for use in therapy.

In an eleventh aspect we provide a binding molecule according to the first aspect, a CAR according to the second aspect, an immune cell, e.g. a cytotoxic immune cell according to the fifth aspect or a pharmaceutical composition according to the sixth aspect for use in the treatment of cancer, wherein the cancer expresses p95HER2.

In a twelfth aspect we provide a method of diagnosing cancer in a subject, the method comprising:
(a) contacting a sample of cells from the subject with a binding molecule as defined herein, wherein the binding molecule further comprises a detection moiety;
(b) determining whether the cells express p95HER2; and
(c) if the cells express p95HER2, diagnosing the subject (e.g. patient) with cancer.

The method of the twelfth aspect is thus an ex vivo method performed on a sample.

The aspects of the invention above are defined with respect to binding molecules as defined in the first aspect. However, the disclosure herein also extends to other binding molecules, comprising antigen binding units based on other variants of the variable domains of the Oslo-2 antibody, as also described in the Examples below. Thus, also provided herein are aspects as set above, relating to binding molecules, CARs, nucleic acids, vectors, immune cells, pharmaceutical compositions, medical uses and methods etc., wherein the binding molecule comprises a VH and/or VL which comprises a mutant (i.e. variant) sequence as disclosed below. Reference may be made in this regard to the mutants set out in Table 1 below, and to Mutants 1-3 as disclosed in the Examples below.

In a particular aspect, provided herein is a binding molecule which specifically binds p95HER2 comprising the amino acid sequence set forth in SEQ ID NO: 1, comprising a VL and a VH which together form an antigen binding unit, wherein
the VL comprises three complementarity determining regions (CDRs): LCDR1, LCDR2 and LCDR3 which respectively comprise the amino acid sequences SEQ ID NOs: 16, 3 and 17 (wherein in SEQ ID NO. 16 the residue corresponding to residue 31 of the native VL of antibody Oslo-2 is S31M/W and in SEQ ID NO. 17 the residue corresponding to residue 107 is H107 (the native residue) or H107W); and
the VH comprises three CDRs: HCDR1, HCDR2 and HCDR3 which respectively comprise the amino acid sequences SEQ ID NOs: 5, 6 and 7.

In the aspect above, SEQ ID NO: 16 has the consensus sequence:
VL CDR1: KSSQSLLW/MSGNQKNNLA, wherein the residue shown in bold corresponds to residue 31 of the native VL of SEQ ID NO: 15 and is W or M.
   SEQ ID NO: 17 has the consensus sequence:
VL CDR3: LQH/WYSSPYT, wherein the residue shown in bold corresponds to residue 107 of the native VL of SEQ ID NO: 15 and is H (the native sequence) or W (mutant).

### BRIEF DESCRIPTION OF THE DRAWINGS

FIG. 1: Expression of new antibody variants. FIG. 1 depicts the SDS-PAGE gel electrophoresis results for the two new antibody variants (variant 4 and variant 5) for the purpose of quality control.

FIG. 2: Antibody binding capacity. FIG. 2 shows the absorbance at 450 nm at increasing antibody concentrations of the control antibody, the Oslo-2 parental antibody, variant 4 and variant 5 antibodies.

### DETAILED DESCRIPTION

Binding molecules herein are derived from the antigen binding domain of the antibody Oslo-2. The CDRs of the parental Oslo-2 antibody, according to the Kabat nomenclature, and as disclosed in PCT/EP2022/079110, are set out below:

VL CDR1 (SEQ ID NO: 13):
KSSQSLLSSGNQKNNLA
VL CDR2 (SEQ ID NO: 3):
YASTRQS
VL CDR3 (SEQ ID NO: 9):
LQHYSSPYT
VH CDR1 (SEQ ID NO: 5):
DYFMN
VH CDR2 (SEQ ID NO: 6):
QIRNKNYNYATYFAESLEG
VH CDR3 (SEQ ID NO: 7):
LRYDY

The amino acid sequence of the VH of the parental antibody is shown in SEQ ID NO: 10, in which the 3 CDRs are boxed:

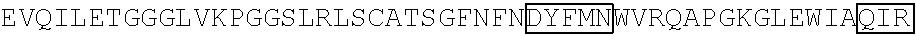

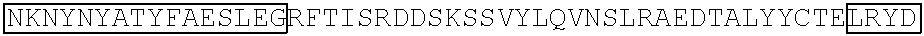

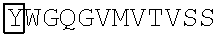

The amino acid sequence of the VL of the parental antibody is shown in SEQ ID NO: 15, in which the 3 CDRs are boxed:

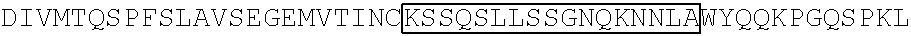

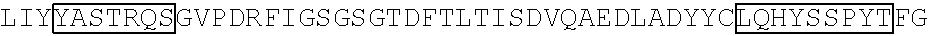

AGTKLELK

In the antigen binding units of the binding molecules herein, mutations have been introduced in CDR1, and optionally CDR3, of the light chain variable domain.

Specifically, in one variant of the antigen binding unit of the binding molecule, identified herein as Mutant 4, the VH CDRs are unchanged and the VL CDRs, according to the Kabat nomenclature, are as follows:

VL CDR1 (SEQ ID NO: 2):
KSSQSLLWSGNQKNNLA
VL CDR2 (SEQ ID NO: 3):
YASTRQS
VL CDR3 (SEQ ID NO: 4):
LQWYSSPYT

The mutations S31W CDR1 and H107W in VL are shown in bold.

In another variant of the antigen binding unit of the binding molecule, identified herein as Mutant 5, the VH CDRs are unchanged and the VL CDRs, according to the Kabat nomenclature, are as follows:

VL CDR1 (SEQ ID NO: 8):
KSSQSLLMSGNQKNNLA
VL CDR2 (SEQ ID NO: 3):
YASTRQS
VL CDR3 (SEQ ID NO: 9):
LQHYSSPYT

The mutation S31M in CDR1 is shown in bold.

Binding molecules comprising the novel antigen binding units herein generally comprise or consist of one or more proteins (i.e. polypeptide chains) and may have any suitable format including antibodies, which term includes all antibody formats and fragments, including, for example, scFv's, Fab's, immunotoxins, immunoconjugates, bispecific antibodies, CARs etc.

Thus, in an embodiment the binding molecule provided herein is an antibody or a fragment (that is, an antigen-binding fragment) thereof. Examples of antigen binding fragments of antibodies include Fab, Fab′ and F(ab)′₂ moieties. In another embodiment the binding molecule is a scFv. In still another embodiment the binding molecule is a CAR.

In yet another embodiment, the binding molecule is in the form of a conjugate with another moiety. More particularly, the conjugate may comprise the binding molecule, or more particularly, the antigen binding unit of the binding molecule as defined herein, linked, directly or indirectly, to one or more other moieties. The other moiety may be a therapeutic or diagnostic agent, or another binding domain, for example a different binding molecule comprising an antigen binding unit having a different specificity. Thus, the binding molecule may be in the form of bi- or tri, or higher order multi-specific binding molecule, for example, a bi- or tri-specific antibody. In an embodiment, the binding molecule is a bi-specific T-cell engager (BiTE).

In the conjugates, the binding molecule can be an antibody in any format described herein, for example a full length antibody or a fragment thereof, or a scFv etc.

The binding molecule in the conjugate may be linked directly via a bond, or indirectly via a linker or linking group. The bond may be a peptide bond and the linker may be a peptide, in which case the conjugate is in the form of a fusion protein. However, in other embodiments, a chemical linking group may be used to link one or more other moieties to the binding molecule.

The binding molecule provided herein specifically binds p95HER2 comprising the amino acid sequence PIWKFPDEE as set forth in SEQ ID NO: 1. As further detailed below, SEQ ID NO: 1 is the epitope recognised by the binding molecules provided herein.

Such binding molecules, especially soluble binding molecules such as antibodies, antigen-binding fragments of antibodies and scFvs, may be used in their “naked” form (i.e. not conjugated to a second agent) to target cancer cells. Alternatively, such binding molecules may carry (e.g. be conjugated to) a toxic payload, e.g. a cytotoxin (such as saporin or gelonin) or a moiety comprising a radioactive isotope such as ¹⁷⁷Lu, ²²⁴Ra or ²²⁵Ac. A binding molecule conjugated to a toxic payload may be referred to as an immunotoxin. The terms “toxin” and “toxic payload” are used broadly herein to include any entity (i.e. compound or substance) having a toxic effect on mammalian cells, notably a cytotoxic effect. Thus, the toxin may be any agent which inhibits the growth and/or viability of mammalian cells, and in particular cancer cells. It may be any agent having cytotoxic activity.

More broadly, the conjugate may comprise the binding molecule (or the antigen binding unit thereof) linked to a therapeutic agent effective in the treatment of cancer. This may be any chemotherapeutic agent, and more particularly any chemotherapeutic agent used or effective in the treatment of any cancer, or for treatment of HER2-positive cancer. The therapeutic agent may be any agent having a therapeutic effect, or in other words, any drug. Such conjugates may accordingly be referred as antibody drug conjugates (ADC). The use of ADC in cancer therapy is well known in the art. Any drug known or used in ADC as known or described in the art may be used in the conjugates herein.

Examples of representative drugs or cytotoxic agents include: Deschloroclozapine (DCZ), Mertansine (DM1), Monomethyl auristatin E (MMAE), Pyrrolobenzodiazepine (PBD), Pyrrolobenzodiazepine Monoamide (PBD-MA), Auristatin F-hydroxypropylamide (AF-HPA), DM4, Seco-duocarmycinhydroxybenzamide-azaindole (Seco-DUBA), the 7-ethyl-10-hydroxycamptothecin (SN-38), AZI13599185, and AS269 (Amberstatin).

Furthermore, the novel antigen binding units may be used as diagnostic agents, e.g. in the form of naked antibodies or binding molecules comprising a detectable label like a fluorescent or radioactive moiety. A detectable label may be referred to as a detection moiety, or a moiety suitable for detection. Such conjugates comprising the binding molecule, or antigen binding unit thereof, linked to a diagnostic agent may be for in vitro, or in vivo diagnostic use. Thus, the detection moiety may be a moiety suitable for detection in vivo, e.g. by imaging, for example a CT and PET or SPECT tracer or a contrast agent, or imaging agent, e.g. detectable by MRI.

In another embodiment the binding molecule is in the form of a BiTE. BiTEs are known and described in the art for cancer therapy. The BiTE may be defined as a bispecific binding molecule comprising a binding molecule of the invention as defined herein linked to a second binding molecule capable of binding specifically to a T-cell. Analogously, bi-specific binding molecules may be generated which are capable of engaging other immune cells, for example NK cells, macrophages or monocytes. Accordingly, the binding molecule may be more generally provided in the form of a bi-specific immune cell engager, defined as comprising a binding molecule of the invention as defined herein, linked to a second binding molecule capable of binding to an immune cell. The second binding molecule may comprise an antigen binding unit derived from an antibody capable of binding specifically to an immune cell, e.g. a T-cell or NK cell. The binding molecules comprised in such bi-specific engager molecules may be in the form of scFVs linked together by a linker sequence.

In a typical BiTE the second binding molecule is directed against CD3. However other targets may be used, for example the second binding molecule may bind to other cell surface markers specific to T-cells or other immune cells, e.g. to NK cell markers. Bi- or tri-specific antibodies etc. may be designed analogously, depending on the target of the second and optionally further binding molecules. Multi-specific antibodies may or may not comprise an Fc region.

As described in PCT/EP2022/079110, the parental Oslo-2 antibody in IgG format has been determined to have a low equilibrium dissociation constant (KD=2 nM), with a high maximal binding response (Rmax) at 137 RU. In an embodiment, the binding molecules herein, in comparable format, exhibit binding of a similar order. However, this is not an absolute requirement; binding affinity of antibodies and binding molecules can vary depending on testing method etc., and thus it is not precluded that in certain assays the binding molecules may have a lower binding affinity compared to the Oslo-2 antibody. A reduced binding affinity may be tolerated so long as it is sufficient for the binding molecule to bind to its target effectively, and indeed in some cases a reduced affinity may be compensated by improvements in other parameters, such as improved stability, or improved production and developability etc., e.g. improved expression yield in host cells etc. Such compensation may not be needed.

In an embodiment, the antigen binding units herein display little or no binding to full length HER2 under physiological conditions and they display little or no cross-reactivity to healthy tissue.

The target epitope of the antigen binding units herein, is believed to be the sequence PIWKFPDEE (SEQ ID NO: 1). Said epitope is located in the p95HER2 isoform called 611-HER2-CTF (SEQ ID NO: 18).

(SEQ ID NO: 18)
MPIWKFPDEEGACQPCPINCTHSCVDLDDKGCPAEQRASPLTSII
SAVVGILLVVVLGVVFGILIKRRQQKIRKYTMRRLLQETELVEPL
TPSGAMPNQAQMRILKETELRKVKVLGSGAFGTVYKGIWIPDGEN
VKIPVAIKVLRENTSPKANKEILDEAYVMAGVGSPYVSRLLGICL
TSTVQLVTQLMPYGCLLDHVRENRGRLGSQDLLNWCMQIAKGMSY
LEDVRLVHRDLAARNVLVKSPNHVKITDFGLARLLDIDETEYHAD
GGKVPIKWMALESILRRRFTHQSDVWSYGVTVWELMTFGAKPYDG
IPAREIPDLLEKGERLPQPPICTIDVYMIMVKCWMIDSECRPRFR
ELVSEFSRMARDPQRFVVIQNEDLGPASPLDSTFYRSLLEDDDMG
DLVDAEEYLVPQQGFFCPDPAPGAGGMVHHRHRSSSTRSGGGDLT
LGLEPSEEEAPRSPLAPSEGAGSDVFDGDLGMGAAKGLQSLPTHD
PSPLQRYSEDPTVPLPSETDGYVAPLTCSPQPEYVNQPDVRPQPP
SPREGPLPAARPAGATLERPKTLSPGKNGVVKDVFAFGGAVENPE
YLTPQGGAAPQPHPPPAFSPAFDNLYYWDQDPPERGAPPSTFKGT
PTAENPEYLGLDVPV

Accordingly, we provide antigen binding units able to specifically bind to the sequence PIWKFPDEE (SEQ ID NO: 1) under physiological conditions. In an embodiment, a binding molecule, e.g. an antibody, comprising an antigen binding unit as provided herein has a KD of at least 2 nM.

As used herein, an “antigen binding unit” is a moiety comprising or consisting of one or more proteins, or parts thereof, able to bind an extracellular target epitope under physiological conditions. The antigen binding units herein may be able to bind an extracellular target epitope under physiological conditions in a tumor environment. The antigen binding units herein can specifically bind to p95HER2 expressed on cancer cells under physiological conditions. That is, the antigen binding units herein display little or no binding to full length HER2 under physiological conditions. Furthermore, the antigen binding units herein display little or no cross-reactivity to healthy tissue.

In particular, the antigen binding units herein can bind to epitopes that are masked in full-length HER2, but are exposed in 611-CTF. This makes them highly specific against the hyperactive p95HER2 isoform. Notably, 611-CTF is the only known isoform of p95HER that extensively induces expression of genes involved in metastasis and development of malignancy.

Binding molecules comprising the antigen binding units provided herein thus specifically bind p95HER2 comprising the amino acid sequence set forth in SEQ ID NO: 1, and can thus bind (or target) cancer cells which express p95HER2 isoforms which comprise the epitope of SEQ ID NO: 1 (such as p95HER2-611-CTF). In particular, the CARs provided herein, which comprise such an antigen binding unit, can target cytotoxic cells expressing the CARs against such cancer cells in order to destroy them.

The antigen binding unit provided herein comprises an antibody light chain variable domain (VL) and an antibody heavy chain variable domain (VH). Such variable domains are well-known for skilled persons. The antigen binding unit of an antibody, comprising a VL and a VH, is called a Fv. An antigen binding unit as defined herein may comprise a single polypeptide chain comprising both the VL and VH sequences (e.g. as in the case of an scFv), or alternatively the VL and VH may be provided on separate polypeptide chains (as in an Fv).

Each VL and VH herein comprises three complementarity determining regions (CDRs) flanked by framework sequences. The framework sequences may be human, humanized or murine sequences. In the binding molecules of the invention the three VH CDRs comprise or consist of SEQ ID NO:s 5, 6, 7 as defined for the parental antibody above. The three VL CDRs comprise or consist of the following sequences, as set out above:

(SEQ ID NO: 2)
VL CDR1;
(SEQ ID NO: 3)
VL CDR2;
(SEQ ID NO: 4)
VL CDR3;
or
(SEQ ID NO: 8)
VL CDR1;
(SEQ ID NO: 3)
VLCDR2;
(SEQ ID NO: 9)
VL CDR3.

A Framework1 sequence is N-terminal to the CDR1, a Framework2 sequence is located between CDR1 and CDR2, while a Framework3 sequence is located between CDR2 and CDR3.

Accordingly, both a VL and VH can be roughly visualized as follows, with the CDRs boxed and the N-terminus indicated as N-:

N-

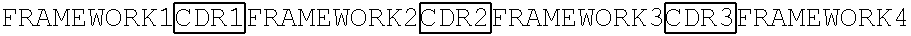

RK4

In one embodiment, the antigen binding unit comprises a murine VH comprising or consisting of the following sequence, in which the three CDRs are boxed (SEQ ID NO: 10):

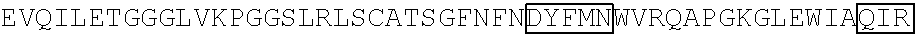

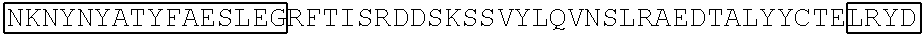

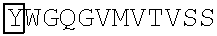

In another embodiment, the antigen binding unit comprises a VH comprising or consisting of a sequence with at least 80%, 85%, 90% or 95% identity to SEQ ID NO: 10.

In one embodiment, the antigen binding unit comprises a VL comprising or consisting of the following sequence, in which the three CDRs are boxed (SEQ ID NO: 11):

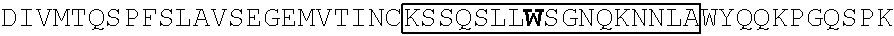

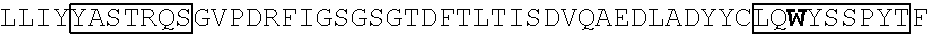

GAGTKLEIK

In another embodiment, the antigen binding unit comprises a VL comprising or consisting of a sequence with at least 80%, 85%, 90% or 95% identity to SEQ ID NO: 11.

In one embodiment, the antigen binding unit comprises a VL comprising or consisting of the following sequence, in which the three CDRs are boxed (SEQ ID NO: 12):

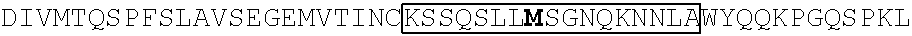

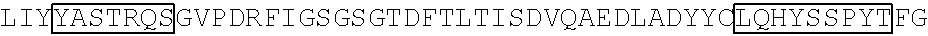

AGTKLEIK

In another embodiment, the antigen binding unit comprises a VL comprising or consisting of a sequence with at least 80%, 85%, 90% or 95% identity to SEQ ID NO: 12.

In SEQ ID NO:s 11 and 12 the VL sequence also comprises a L to I substitution in the penultimate residue (L112I), compared to the VL sequence of the parental antibody as shown in SEQ ID NO:15. The parental Oslo-2 antibody is a rat IgG2a antibody, and the VL sequences of the new variants have been modified to allow reformatting to mouse IgG1 format.

The VH and VL may be connected by a disulphide bridge or a peptide linker. Alternatively, the two chains may be located within a Fab-fragment of an antibody (or any other antigen-binding fragment of an antibody) or an antibody as such. In one embodiment, the antigen binding unit comprises or consists of VL-linker-VH (from N- to C-terminus). In another embodiment, the antigen binding unit comprises or consists of VH-linker-VL (from N- to C-terminus). Such antigen binding units are often referred to as single chain Fv-fragments (scFv's). The linker has to have a certain length in order to allow the VH and VL to form a functional antigen binding unit. In one embodiment, the linker comprises 10 to 30 amino acid residues. In one embodiment, the linker comprises 15 to 25 amino acid residues, in particular glycine and/or serine residues.

In a particular embodiment the linker is a G₄S linker, i.e. a peptide linker comprising repeating units with the sequence GGGGS (SEQ ID NO: 19). For instance, the linker may be a (G₄S)₃ (SEQ ID NO: 20), (G₄S)₄ (SEQ ID NO: 21) or (G₄S)₅ (SEQ ID NO: 22) linker (i.e. a linker comprising 3, 4 or 5 adjoining repeating G₄S units, respectively).

The linker may alternatively be a modified G₄S linker comprising one or more amino acid substitutions (optionally conservative amino acid substitutions, as defined below) in one or more G₄S units (preferably up to one amino acid substitution in one or more G₄S unit). In particular, a modified G₄S unit may comprise one or more substitutions of alanine for glycine. An example of a suitable linker as demonstrated below has the amino acid sequence set forth in SEQ ID NO: 23, which is a modified (G₄S)₄ linker in which one glycine residue has been substituted for alanine:

(SEQ ID NO: 23)
GGGGSGGGGSAGGGSGGGGS

In antigen binding units, the framework sequences may tolerate variation without destroying the specificity and affinity to the target antigen. For example, substitutions of amino acid residues may be tolerated better than deletions or additions of amino acid residues. Replacing murine framework sequences with human framework sequences, preferably of similar length, is known as humanization. Variants of SEQ ID NO:s 10, 11 and 12 are included herein, which have at least 80, 85, 90 or 95% sequence identity to SEQ ID NOs 10, 11 or 12, and which comprise one or more amino acid modifications to the framework regions thereof (that is the CDR sequences are not modified). In particular variants with humanized framework regions are included.

The term “conservative amino acid substitution”, as used herein, refers to an amino acid substitution in which one amino acid residue is replaced with another amino acid residue having a similar side chain.

Amino acids with similar side chains tend to have similar properties, and thus a conservative substitution of an amino acid important for the structure or function of a polypeptide may be expected to affect polypeptide structure/function less than a non-conservative amino acid substitution at the same position. Families of amino acid residues having similar side chains have been defined in the art, including basic side chains (e.g. lysine, arginine, histidine), acidic side chains (e.g. aspartic acid, glutamic acid), uncharged polar side chains (e.g. asparagine, glutamine, serine, threonine, tyrosine), non-polar side chains (e.g. glycine, cysteine, alanine, valine, leucine, isoleucine, proline, phenylalanine, methionine, tryptophan) and aromatic side chains (e.g. tyrosine, phenylalanine, tryptophan, histidine). Thus, a conservative amino acid substitution may be considered to be a substitution in which a particular amino acid residue is substituted for a different amino acid residue in the same family. In particular, the products comprising a conservative amino acid substitution relative to a reference sequence are covered by the terminology.

In one embodiment, each VL and VH herein comprises three CDRs flanked by human framework sequences. Human framework sequences are structurally conserved regions that normally tend to form a β-sheet structure delicately positioning the CDRs for specific binding to the target antigen under physiological conditions. Many human framework sequences are available from known human antibodies and from the the international ImMunoGeneTics information system (IMGT) online database (see Giudicelli et al, Nucleic Acids Research, 2006, Vol. 34, Database issue D781-D784), but the term also covers human framework sequences comprising amino acid substitutions. Each of the human framework sequences may optionally comprise 0 to 5 amino acid substitutions relative to the natural sequence. An amino acid substitution is a sequence wherein an amino acid residue in a specific position is substituted for a different amino acid residue at the corresponding position, apparent when the sequences are aligned. Each of the human framework sequences may optionally comprise 1 amino acid substitution.

Each of the human framework sequences may optionally comprise 2 or up to 2 amino acid substitutions. Each of the human framework sequences may optionally comprise 3 or up to 3 amino acid substitutions. Each of the human framework sequences may optionally comprise 4 or up to 4 amino acid substitutions. Each of the human framework sequences may optionally comprise 5 or up to 5 amino acid substitutions. The substitutions may be conservative substitutions. Even if such framework sequences are not necessarily previously known from human antibodies, they may provide lower immunogenic risk compared to a murine framework sequence. In one embodiment, 0 to 5 amino acid residues in the human framework sequences are substituted with the corresponding amino acid residue(s) from the murine parent sequences found in SEQ ID NOs: 10 and 15.

Collectively, scFv's comprising CDRs from a murine antibody and human framework sequences which each may optionally comprise 0 to 5 substitutions, are referred to as humanized scFv's. In some embodiments, some of the substitutions may be back to the parent murine amino acid residue (also known as “back mutations”).

In one embodiment, the human framework sequences are mature human framework sequences available from known human antibodies. Without being bound by theory, such framework sequences may convey very low risk of triggering unwanted immunogenic responses against the antigen binding unit, and at the same time increase the likelihood of obtaining stable binding units which are expressed well in cellular systems.

Generally speaking, in humanised VH and VL sequences the CDRs are not altered, and are retained, as in the parental VH and VL sequences. However as is known in the art, different programs, or schemes, are available to determine CDR sequences, and these may not in all cases give exactly co-incident results. Thus, different CDR identification schemes may yield different CDR sequences. For example, they may be shorter or longer, or positioned slightly differently in the VH or VL sequences (e.g. in a second scheme the CDR sequence may be partially displaced up- or downstream relative to a first scheme). Humanisation may be performed using a CDR grafting algorithm which uses different versions of the identified CDRs to transfer the CDRs from the original framework onto selected human sequences. Thus, a humanised sequence may contain CDRs as identified according to any of the CDR identification schemes, e.g. the Kabat scheme, the IMGT scheme, and the Chothia scheme.

In certain representative embodiments, the antigen binding unit is or comprises an scFv comprising or consisting of the following sequences, from N to C terminal:
(i) The VH sequence of SEQ ID NO: 10 or a sequence with at least 80% sequence identity thereto, the linker sequence of SEQ ID NO: 23, and the VL sequence of SEQ ID NO: 11 or a sequence with at least 80% sequence identity thereto; or
(ii) The VH sequence of SEQ ID NO: 10 or a sequence with at least 80% sequence identity thereto, the linker sequence of SEQ ID NO: 23, and the VL sequence of SEQ ID NO: 12 or a sequence with at least 80% sequence identity thereto; or
(iii) The VL sequence of SEQ ID NO: 11 or a sequence with at least 80% sequence identity thereto, the linker sequence of SEQ ID NO: 23, and the VH sequence of SEQ ID NO: 10 or a sequence with at least 80% sequence identity thereto; or
(iv) The VL sequence of SEQ ID NO: 12 or a sequence with at least 80% sequence identity thereto, the linker sequence of SEQ ID NO: 23, and the VH sequence of SEQ ID NO: 10 or a sequence with at least 80% sequence identity thereto.

The antibody, as noted above, may take various forms, including antibody fragments. All such forms are included. The antibody may comprise one or more antigen-binding domains, or one or more VH and/or one or more VL regions. The VH and/or VL regions may be comprised in a single chain (polypeptide) or on separate chains (polypeptides). The antibody may accordingly comprise one or more polypeptide chains, e.g. 2 or 4 polypeptides. An individual polypeptide may comprise one or more VH and/or more one or more VL regions, e.g. both a VH and a VL region etc.

Accordingly, an antigen-binding domain may comprise one or more polypeptides (chains), each comprising one or more VH and/or one or more VL regions.

Further, a polypeptide (chain) comprising a variable region sequence may comprise all or part of a constant region sequence, for example one, two or all three of CH1, CH2 and CH3 of a heavy chain constant region in the case of VH, and all or part of the light chain constant region (CL) in the case of VL.

Thus, the term “antibody” includes all known forms of antibody, including whole, or full-length, antibodies, or any antigen-binding fragments thereof, or single chains or single-chain derivatives thereof, as well as synthetic or artificial antibody constructs which comprise at least one VH and at least one VL region as defined herein, and multimers thereof, e.g. dimeric, trimeric or higher order multimeric antibodies. It will be understood that the term includes recombinant and engineered antibodies.

More broadly, the term “antibody” can be seen to include any binding protein (which may be referred to as an immunological binding protein) which comprises an antigen-binding domain, specifically an antigen-binding domain derived from an antibody. Accordingly, the term “antibody” is thus used to refer to any antibody-like molecule that has an antigen binding region obtained or derived from an antibody. The antibody or fragment thereof comprises at least one VH region and a least one VL region.

In one embodiment, the antibody is an immunoglobulin antibody, and more specifically an antibody comprising at least 2 heavy chains and at least 2 light chains, or a fragment thereof.

In any format herein, wherein the antibody comprises constant regions, the heavy chain comprises the VH herein and all or part of a heavy chain constant region and the light chain comprises the VL herein and all or part of a light chain constant region. When the antibody comprises a full complement of constant regions from the heavy and light chains it is referred to as a whole antibody, or full-length antibody. Such full-length/whole antibodies represent one preferred embodiment. Depending on the type of constant domain in the heavy chains, antibodies are assigned to one of five major classes: IgA, IgD, IgE, IgG, and IgM and any of these are included, although IgA and IgG are preferred, particularly IgG. Several of these are further divided into subclasses or isotypes, such as IgG1, IgG2, IgG3, IgG4, and the like, for example camelid antibodies are IgG antibodies which often have IgG2 or IgG3 constant domains. All sub-classes are included herein. The heavy-chain constant domains that correspond to the difference classes of immunoglobulins are termed α, δ, ε, γ and μ, respectively. The subunit structures and three-dimensional configurations of different classes of immunoglobulins are well known. Suitable heavy chain constant regions are known and available in the art.

The light chains of mammalian antibodies are assigned to one of two clearly distinct types: kappa (κ) and lambda (λ), and either of these may be used. Again, suitable light chain constant region sequences are known and available in the art.

In an embodiment the heavy chain constant region is or comprises all or part of the mouse IgG1 constant region having the amino acid sequence set forth in SEQ ID NO: 24 or an amino acid sequence having at least 90% sequence identity thereto.

mouse IgG1 constant region
SEQ ID NO: 24
AKTTPPSVYPLAPGSAAQTNSMVTLGCLVKGYFPEPVTVTWNSGS
LSSGVHTFPAVLQSDLYTLSSSVTVPSSTWPSQTVTCNVAHPASS
TKVDKKIVPRDCGCKPCICTVPEVSSVFIFPPKPKDVLMISLTPK
VTCVVVDISKDDPEVQFSWFVDDVEVHTAQTKPREEQINSTFRSV
SELPILHQDWLNGKEFKCRVNSAAFPAPIEKTISKTKGRPKAPQV
YTIPPPKEQMAKDKVSLTCMITNFFPEDITVEWQWNGQPAENYKN
TQPIMDTDGSYFVYSKLNVQKSNWEAGNTFTCSVLHEGLHNHHTE
KSLSHSPGK,

In another embodiment, the light chain constant region is or comprises all or a part of the mouse kappa light chain constant region having the amino acid sequence set forth in SEQ ID NO: 25 or an amino acid sequence having at least 90% sequence identity thereto.

mouse kappa light chain constant region
SEQ ID NO: 25
RADAAPTVSIFPPSSEQLTSGGASVVCFLNNFYPKDINVKWKIDGS
ERQNGVLNSWTDQDSKDSTYSMSSTLTLTKDEYERHNSYTCEATHK
TSTSPIVKSFNRNEC,

Alternatively, in another embodiment the heavy chain constant region is or comprises all or part of the human IgG1 constant region having the amino acid sequence set forth in SEQ ID NO: 37 or an amino acid sequence having at least 90% sequence identity thereto.

human IgG1 constant region
SEQ ID NO: 37
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK,

In another embodiment, the light chain constant region is or comprises all or a part of the human kappa light chain constant region having the amino acid sequence set forth in SEQ ID NO: 38 or an amino acid sequence having at least 90% sequence identity thereto.

human kappa light chain constant region
SEQ ID NO: 38
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC,

Where the binding molecules are to be used in soluble form, that is not expressed on the surface of cells, e.g. as antibodies and certain conjugates etc., they conveniently may be produced by expression in host cells, e.g. cell lines for production. Such cell lines may conveniently be mammalian cells, such as HEK293 cells for example. Techniques and materials for such expression are well known and widely available in the art. For example, nucleotide sequences encoding variable domains for the binding molecules as defined herein may be designed and codon optimised for expression in mammalian cells. The sequences may be subcloned into cloning and expression vectors. For example, for production of antibodies, vectors are available or known in the art, which comprise sequences for expression of antibodies of desired isotype and subtype (e.g. with appropriate expression control and coding sequences for desired or selected constant region sequences etc. Heavy and light chains may be expressed from separate vectors, or the binding molecule may be expressed from a single vector depending on the selected antibody format etc.

The binding molecule, or individual polypeptide chains thereof may be expressed with a signal peptide. A variety of different signal sequences are known and described in the art.

For example, for expression of VH and VL polypeptides herein a signal peptide of SEQ ID NO: 35 may be used.

SEQ ID NO: 35
MPLLLLLPLLWAGALA

Novel chimeric antigen receptors (CARs) are provided. When the CARs herein are expressed on the surface of immune cells, such immune cells may be used in medicine. In particular, said immune cells may be used in treatment of solid tumors expressing p95HER2 comprising the amino acid sequence set forth in SEQ ID NO: 1. In one embodiment, said immune cells are used in treatment of p95HER2-positive breast cancer, p95HER2-positive gliomas or other p95HER2-positive cancers such as bladder, ovarian, cervical, uterine, prostate, lung, kidney, and colorectal cancers.

As used herein, CARs are artificial receptors comprising an extracellular antigen binding unit, a transmembrane domain and an intracellular signaling domain. The antigen binding unit in CARs is usually a scFv.

The antigen binding unit may be directly attached to the transmembrane domain. However, the CARs may comprise a hinge domain connecting the antigen binding unit to the transmembrane domain. The hinge domain may thus affect the steric conformation of the antigen binding unit. This may in turn affect the ability of the CAR to bind the target epitope and subsequently trigger signaling into an immune cell. If the target epitope is located too far from the cell membrane of the target cell or if the target epitope is otherwise hidden, the immune cell expressing the CAR may not be efficient. Accordingly, it is preferred that the target epitope is sufficiently accessible for immune cells expressing the CARs.

The transmembrane domain connects the extracellular domains to an intracellular signaling domain. Both the antigen binding unit and hinge domain are extracellular domains, i.e. they generally face the extracellular environment when expressed in the cell membrane of an immune cell. As used herein, “transmembrane domain”, means the part of the CAR which tends to be embedded in the cell membrane when expressed by an immune effector cell. Suitable transmembrane domains are well known for skilled persons. In particular, transmembrane domains from the human proteins CD8α, CD28 or ICOS may be used. The transmembrane domain is believed to convey a signal into immune cells upon binding of a target by the antigen binding unit.

The “intracellular signaling domain” refers to a part of the CAR located inside the immune cell when the CAR is expressed in the cell membrane. These domains participate in conveying the signal upon binding of the target. A variety of signaling domains are known, and they can be combined and tailored to fit the endogenous signaling machinery in the immune cells. In one embodiment the intracellular signaling domain comprises a “signal 1” domain like the signaling domains obtainable from the human proteins CD3ζ, FcR-γ, CD3ε etc. In general, it is believed that “signal 1” domains (e.g. the CD3ζ signaling domain) convey a signal upon antigen binding.

In another embodiment, the intracellular signaling domain further comprises a costimulatory domain. Such domains are well known and often referred to as “signal 2” domains, and they are believed to, subsequently to “signal 1” domains, convey a signal via costimulatory molecules. The “signal 2” is important for the maintenance of the signal and the survival of the cells. If absent, like in first-generation CARs, a CAR-T cell may be efficient in killing and in early cytokine release, but it will often become exhausted over time. Thus, the intracellular signaling domain generally comprises both a “signal 1” and “signal 2” domain. Examples of such commonly used human “signal 2” domains include the 4-1BB signaling domain, CD28 signaling domain and ICOS signaling domain.

CARs in the present disclosure may comprise any of the antigen binding units as mentioned above. For example, CARs in the present disclosure may comprise an scFv as described above.

In particular, the CARs in the present disclosure may comprise any of the antigen binding units as mentioned above in the form of a scFv connected to a CD8α hinge. The CD8α hinge is generally the human CD8α hinge of SEQ ID NO: 26, or a variant thereof with at least 90% or 95% sequence identity thereto.

(SEQ ID NO: 26)
SDPTTTPAPRPPTPAPTIASQPLSLRPEACRPAAGGAVHTRGLDF

In particular, the CARs in the present disclosure may comprise an scFv as defined above and an intracellular signaling domain comprising a CD3ζ signaling domain. The CD3ζ signaling domain is generally the human CD3ζ signaling domain of SEQ ID NO: 27, or a variant thereof with at least 90% or 95% sequence identity thereto.

(SEQ ID NO: 27)
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR

In a particular embodiment, in addition to the CD3ζ signaling domain the intracellular signaling domain further comprises a co-stimulatory domain, which may be any such domain as set out above, but in a particular embodiment is a 4-1BB co-stimulatory domain. The 4-1BB co-stimulatory domain is generally the human 4-1BB co-stimulatory domain of SEQ ID NO: 28, or a variant thereof with at least 90% or 95% sequence identity thereto.

(SEQ ID NO: 28)
KRGRKKLLYIFKQPFMRPVQTTQEEDGCSCRFPEEEEGGCEL

The CAR provided herein may in particular comprise a CD8α transmembrane domain, in particular the human CD8α transmembrane domain of SEQ ID NO: 29, or a variant thereof with at least 90% or 95% sequence identity thereto.

(SEQ ID NO: 29)
ACDIYIWAPLAGTCGVLLLSLVITLYC

In a particular embodiment the CAR comprises a CD8α hinge as described above and a CD8α transmembrane domain.

In particular, the CARs in the present disclosure may comprise a scFv as described above connected to a CD8α hinge (SEQ ID NO: 26), wherein the CAR further comprises a CD8α transmembrane domain (SEQ ID NO: 29) and wherein the intracellular signaling domain comprises or consists of a 4-1BB costimulatory domain (SEQ ID NO: 28) and a CD3ζ signaling domain (SEQ ID NO: 27).

Sequence identity may be assessed by any convenient method. However, for determining the degree of sequence identity between sequences, computer programmes that make pairwise or multiple alignments of sequences are useful, for instance EMBOSS Needle or EMBOSS stretcher (both Rice, P. et al., Trends Genet., 16, (6) pp 276-277, 2000) may be used for pairwise sequence alignments while Clustal Omega (Sievers F et al., Mol. Syst. Biol. 7:539, 2011) or MUSCLE (Edgar, R. C., Nucleic Acids Res. 32(5):1792-1797, 2004) may be used for multiple sequence alignments, though any other appropriate programme may be used. Another suitable alignment programme is BLAST, using the blastp algorithm for protein alignments and the blastn algorithm for nucleic acid alignments. Whether the alignment is pairwise or multiple, it must be performed globally (i.e. across the entirety of the reference sequence) rather than locally.

Sequence alignments and % identity calculations may be determined using for instance standard Clustal Omega parameters: matrix Gonnet, gap opening penalty 6, gap extension penalty 1. Alternatively, the standard EMBOSS Needle parameters may be used: matrix BLOSUM62, gap opening penalty 10, gap extension penalty 0.5. Any other suitable parameters may alternatively be used.

The immune cells expressing the CARs herein may be isolated from a patient or a compatible donor by leukapheresis or other suitable methods. Such primary cells may for example be T cells, NK cells or Macrophages. In particular, autologous T cells (both cytotoxic T cells, T helper cells or mixtures of these) may be transduced or transfected with nucleic acids encoding the CARs before a pharmaceutical composition comprising the cells is administered back to the patient. The immune cells expressing the CARs may also be cell lines suitable for clinical use like NK-92 cells. Generally, the immune cell expressing the CAR (whether a primary cell or a cell line) is a T cell (particularly a cytotoxic T cell) or an NK cell. Of course, the preferred cells are human when the intended patient is human.

The pharmaceutical composition herein can be a composition suitable for administration of therapeutic cells to a subject e.g. patient. The most common administration route for CAR T cells is intravenous administration. Accordingly, said pharmaceutical compositions may for example be sterile aqueous solutions with a neutral pH. For example, a subject's peripheral blood mononuclear cells may be obtained via a standard leukapheresis procedure. The mononuclear cells may be enriched for T cells, before transducing or transfecting them with a lentiviral vector or mRNA encoding the CARs. Said cells may then be activated with anti-CD3/CD28 antibody coated beads. The transduced/transfected T cells may be expanded in cell culture, washed, and formulated into a sterile suspension, which can be cryopreserved. If so, the product is thawed prior to administration.

In situations where the tumor is localized, different administrations methods may be used to improve efficacy. For example, regional or local administration rather than systemic administration of CAR-T cells might enhance efficacy.

The pharmaceutical compositions may comprise a pharmaceutically effective dose of the immune cells herein. A pharmaceutically effective dose may for example be in the range of 1×10⁶ to 1×10¹⁰ immune cells expressing the CARs. A pharmaceutically effective dose may for example be in the range of 1×10⁷ to 1×10⁹ T cells expressing the CARs. A pharmaceutically effective dose may for example be in the range of 1×10⁷ to 1×10⁹ NK cells expressing the CARs.

For efficient expression of the claimed CARs in immune cells, a conventional leader peptide may be introduced N-terminally for facilitating location in the cell membrane. One example of a suitable leader peptide is MESQTQALISLLLWVYGTYG (SEQ ID NO:14). The leader peptide is believed to be trimmed off and will likely not be present in the functional CAR in the cell membrane.

Accordingly, for expression of a second-generation CAR, nucleic acids encoding the following may be used:
N-LEADER PEPTIDE-VH-LINKER-VL-HINGE-TRANSMEMBRANE DOMAIN-COSTIMULATORY DOMAIN-SIGNALING DOMAIN. Accordingly, for expression of a second-generation CAR, nucleic acids encoding the following may also be used:
N-LEADER PEPTIDE-VL-LINKER-VH-HINGE-TRANSMEMBRANE DOMAIN-COSTIMULATORY DOMAIN-SIGNALING DOMAIN

The nucleic acids encoding the claimed CARs can be in the form of well-known RNA e.g. mRNA, or DNA expression vectors.

The pharmaceutical composition provided herein may alternatively be a composition suitable for administration of the binding molecule provided herein (e.g. antibody) to a subject, e.g. patient. Such a composition generally comprises one or more pharmaceutically-acceptable excipients or suchlike, which are known in the art. A binding molecule as provided herein (such as an antibody), or a pharmaceutical composition comprising such a binding molecule, may be used in medicine/therapy, in particular to treat cancer expressing p95HER2 comprising the amino acid sequence set forth in SEQ ID NO: 1. The binding molecule or pharmaceutical composition may in particular be used to treat a solid cancer, e.g. breast cancer or glioma.

The subject may be any human or non-human animal, particularly any mammalian animal, and this may include domestic, farm, livestock, sports, or zoo animals etc. Particularly, the subject is a human, and accordingly the methods and uses herein are particularly for human patients.

In one particular embodiment, there is provided a method of treatment of p95HER2 positive cancer in a subject, e.g. human patient, comprising the steps:
a. transducing or transfecting T cells, NK cells or Macrophages with mRNA encoding any of the CARs herein;
b. repeatedly administering an effective dose of a pharmaceutical composition comprising said cells to a subject, e.g. patient, diagnosed with p95HER2 positive cancer.

In one particular embodiment, there is provided a method of treatment of p95HER2 positive cancer in a subject, e.g. human patient, comprising the steps:
a. transducing T cells, NK cells or Macrophages with mRNA encoding a p95HER2 CAR;
b. repeatedly administering an effective dose of a pharmaceutical composition comprising said cells to a subject, e.g. patient, diagnosed with p95HER2 positive cancer.

In one particular embodiment, a method of treating a subject, e.g. patient, diagnosed with breast cancer is provided, wherein the method comprises the steps:
a. obtaining a sample comprising cancer cells from the subject, e.g. patient;
b. analysing whether the cancer cells express p95HER2; and
c. administering a pharmaceutical composition comprising a pharmaceutically effective dose of T cells, NK cells or Macrophages expressing any of the CARs disclosed herein if the cancer cells are p95HER2 positive.

In one particular embodiment, a method of treating a subject, e.g. patient, diagnosed with breast cancer is provided, wherein the method comprises the steps:
a. obtaining a sample comprising cancer cells from the subject, e.g. patient;
b. analysing whether the cancer cells express p95HER2; and
c. administering a pharmaceutical composition comprising a pharmaceutically effective dose of T cells, NK cells or Macrophages expressing a p95HER2 CAR if the cancer cells are p95HER2 positive.

In one particular embodiment, a method of treating a subject, e.g. patient, diagnosed with breast cancer is provided, wherein the method comprises the steps:
a. obtaining a sample comprising cancer cells from the subject, e.g. patient;
b. analysing whether the cancer cells express p95HER2 by contacting the cells ex vivo with an antibody comprising a VL and a VH as described herein; and
c. administering a cancer therapy, e.g. chemotherapy to the subject, e.g. patient if the cancer cells are p95HER2 positive.

As noted above, the chemotherapy may be an approved chemotherapy. The chemotherapy may specifically target p95HER2 or cells expressing it.

In one particular embodiment, a method of diagnosing cancer in a subject, e.g. human patient is provided, wherein the method comprises the steps
a. obtaining a sample comprising cells from the subject, e.g. patient;
b. analysing whether the cells express p95HER2 by contacting the cells ex vivo with a binding molecule as provided herein, wherein the binding molecule comprises a moiety suitable for detection; and
c. diagnosing the subject, e.g. patient, with cancer if the cells express p95HER2.

In all the preceding aspects and embodiments, unless specified otherwise, the CDRs are identified using the Kabat scheme, and are as specified above. However, as noted above in other aspects and embodiments, the binding molecules may comprise an antigen binding unit comprising a variant VH and/or VL sequence comprising mutations as specified in Table 1 below, or any combination of any of the specified mutations. The disclosures above relating to the detail of the invention may be applied analogously to such aspects and embodiments.

In particular, the binding molecule may comprise an antigen binding unit wherein the VH and VL are defined as follows:
(i) the VL comprises three complementarity determining regions (CDRs): LCDR1, LCDR2 and LCDR3 which respectively comprise the amino acid sequences SEQ ID NOs: 2, 3 and 4; and
the VH comprises three CDRs: HCDR1, HCDR2 and HCDR3 which respectively comprise the amino acid sequences SEQ ID NOs: 5, 6 and 30 (WRDYY) (wherein SEQ ID NO: 30 comprises mutation L107W), identified as Mutant 1 herein; or
(ii) the VL comprises three complementarity determining regions (CDRs): LCDR1, LCDR2 and LCDR3, which respectively comprise the amino acid sequences SEQ ID NOs: 31 (KSSQSLLWSGNQKNMLA) (wherein SEQ ID NO: 31 comprises mutations S31W and N38M), 3 and 32 (LQHDWSPYT) (wherein SEQ ID NO: 32 comprises mutations Y108D and S109W); and
the VH comprises three CDRs; HCDR1, HCDR2 and HCDR3, which respectively comprise the amino acid sequences SEQ ID NOs: 5, 6 and 30, identified as Mutant 2 herein; or
(iii) the VL comprises three complementarity determining regions (CDRs): LCDR1, LCDR2 and LCDR3, which respectively comprise the amino acid sequences SEQ ID NOs: 8, 3 and 9; and
the VH comprises three CDRs; HCDR1, HCDR2 and HCDR3, which respectively comprise the amino acid sequences SEQ ID NOs: 5, 6 and 30, identified as Mutant 3 herein.

Mutants 1, 2 and 3 comprise a mutation (L017W) in the VH (specifically in CDR3), in addition to mutated VL sequences. Mutant 1 comprises the same VL mutations in CDRs 1 and 3 as Mutant 4 herein. Mutant 3 comprises the same VL mutation in CDR1 as mutant 5 herein. Mutant 2 comprises an additional VL mutation in CDR1 compared to Mutant 4, and also alternative mutations in CDR3.

In an embodiment, said binding molecule may comprise a VH comprising the amino acid sequence set forth in SEQ ID NO: 33 or a sequence with at least 80, 85 or 90% identity thereto, and a VL comprising the amino acid sequence set forth in SEQ ID NO: 11 or a sequence with at least 80, 85 or 90% identity thereto (corresponding to Mutant 1).

In another embodiment, said binding molecule may comprise a VH comprising the amino acid sequence set forth in SEQ ID NO: 33 or a sequence with at least 80, 85 or 90% identity thereto, and a VL comprising the amino acid sequence set forth in SEQ ID NO: 34 or a sequence with at least 80, 85 or 90% identity thereto (corresponding to Mutant 2).

In another embodiment, said binding molecule may comprise a VH comprising the amino acid sequence set forth in SEQ ID NO: 33 or a sequence with at least 80, 85 or 90% identity thereto, and a VL comprising the amino acid sequence set forth in SEQ ID NO: 12 or a sequence with at least 80, 85 or 90% identity thereto (corresponding to Mutant 3).

mutant VH comprising L107W
SEQ ID NO: 33
EVQILETGGGLVKPGGSLRLSCATSGFNFNDYFMNWVRQAPGKGL
EWIAQIRNKNYNYATYFAESLEGRFTISRDDSKSSVYLQVNSLRA
EDTALYYCTE [symbol] RYDYWGQGVMVTVSS
SEQ ID NO: 34
mutant VL comprising S31W, N38M, Y108D and S109W
DIVMTQSPFSLAVSEGEMVTINCKSSQSLL [symbol] SGNQKN [symbol] LAWYQQK
PGQSPKLLIYYASTRQSGVPDRFIGSGSGTDFTLTISDVQAEDLA
DYYCLQH [symbol] SPYTFGAGTKLEIK

### EXAMPLES

### Example 1: Antibody Design

### Methods:

In the first step, the structure of the Oslo-2 antibody-p95HER2 complex was determined through docking studies. 10 docked structures were generated using information-driven docking and a consensus structure was found based on epitope overlap. FoldX analysis also showed that the interaction energy between antibody and p95HEr2 antigen to be favorable (−10.87 kcal/mol). In-silico approaches were then used to improve the binding affinity and developability of the antibody.

This included mutational analysis for the Oslo-2 antibody paratope and computational mutation of paratope residues, with the aim of finding 1) important residues for binding and 2) mutations that might potentially improve binding. Various point mutations were identified in this way, leading to the identification of significant point mutations which significantly improved binding affinity (<−1.5 kcal/mol) with high confidence.

There were a total 32 mutations with change in interaction energy less than −1.5 kcal/mol at 6 mutation sites resulting in 35279 antibody variants. Most of these mutations were in the light chain of the antibody.

As expected, we observed a Normal distribution for the mutations. We are interested in the outliers in the far left in the number scale with significant improvement in interaction energy. However, it is also important to note that increasing the number of mutation sites will likely improve the binding significantly. However, more mutations can also lead to destabilization of CDR conformation and in turn loss of binding. Therefore, the objective was to find the best interaction energy with the least number of mutations.

Further study of the top 5 mutations from different numbers of mutation sites and mutation frequency allowed us to identify key frequent mutations.

Therapeutic Antibody Profiler (TAP) was then used to analyse the developability of the antibodies. Table 1 below lists the mutations analysed. We found that “patches of surface hydrophobicity (PSH)” is the most critical parameter, which needs optimization for the variants of antibodies. Mutants were identified with lower PSH scores.

TABLE 1
Mutations
H:L107W
L:S31W
L:S109F
L:S109W
L:H107W
H:L107W/L:S31W
H:L107W/L:S109F
H:L107W/L:H107W
H:L107W/L:S109W
H:L107W/L:S31M
H:L107W/L:S31L/L:H107W
H:L107W/L:S31W/L:N38F
H:L107W/L:S31W/L:S109L
H:L107W/L:S31W/L:S109F
H:L107W/L:S31W/L:H107W
H:L107W/L:S31W/L:H107W/L:S109F
H:L107W/L:S31M/L:H107W/L:S109F
H:L107W/L:S31W/L:Y108E/L:S109F
H:L107W/L:S31W/L:Y108D/L:S109W
H:L107W/L:S31W/L:H107W/L:S109L
H:L107W/L:S31W/L:H107W/L:Y108E/L:S109W
H:L107W/L:S31W/L:H107W/L:Y108D/L:S109W
H:L107W/L:S31W/L:H107W/L:Y108D/L:S109F
H:L107W/L:S31W/L:H107W/L:Y108H/L:S109F
H:L107W/L:S31W/L:N38M/L:Y108D/L:S109W
H:L107W/L:S31W/L:N38H/L:H107W/L:Y108D/L:S109W
H:L107W/L:S31W/L:N38M/L:H107W/L:Y108D/L:S109W
H:L107W/L:S31W/L:N38D/L:H107W/L:Y108D/L:S109W
H:L107W/L:S31W/L:N38H/L:H107W/L:Y108E/L:S109W
H:L107W/L:S31W/L:N38M/L:H107K/L:Y108D/L:S109W

Further in silico aggregation analysis was then performed to identify mutations which reduce the aggregation capability of Aggregation Prone Regions (APRs) in the antibodies.

### Results:

The in silico analysis led to the selection of 5 antibody mutants, identified herein as Mutants 1 to 5. The VH and VL sequences of the Mutants 1-5 (also referred to as Variants 1 to 5) are shown below.

### A. In Both Heavy and Light Chains:

1. H:L107W/L:S31W/L:H107W: Performs well for all developability parameter and APR

Heavy:
(SEQ ID NO: 33)
EVQILETGGGLVKPGGSLRLSCATSGFNFNDYFMNWVRQAPGKGLEWIAQIR

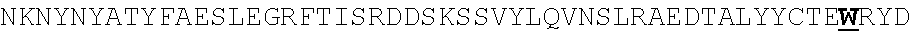

YWGQGVMVTVSS
Light:
(SEQ ID NO: 11)

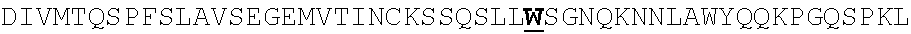

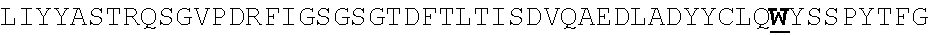

AGTKLEIK

2. H:L107W/L:S31W/L:N38M/L:Y108D/L:S109W: Although PSH score is slightly above the amber flag range in TAP method. It shows least aggregation in ANuPP profile.

Heavy:
(SEQ ID NO: 33)
EVQILETGGGLVKPGGSLRLSCATSGFNFNDYFMNWVRQAPGKGLEWIAQIR

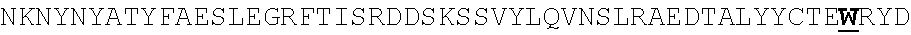

YWGQGVMVTVSS
Light:
(SEQ ID NO: 34)

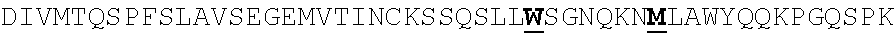

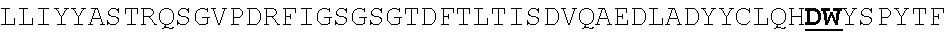

GAGTKLEIK

3. H:L107W/L:S31M: The performance is best with the least number of mutations.

Heavy:
(SEQ ID NO: 33)
EVQILETGGGLVKPGGSLRLSCATSGFNFNDYFMNWVRQAPGKGLEWIAQIR

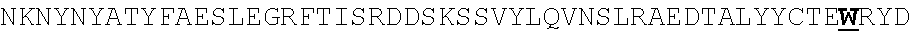

YWGQGVMVTVSS
Light:
(SEQ ID NO: 12)

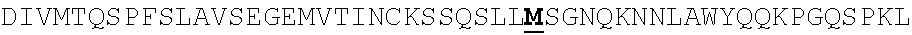

LIYYASTRQSGVPDRFIGSGSGTDFTLTISDVQAEDLADYYCLQHYSSPYTFG
AGTKLEIK

### B. Only in the Light Chain:

4. L:S31W/L:H107W: Performs well for all developability parameter and APR

Heavy:
(SEQ ID NO: 10)
EVQILETGGGLVKPGGSLRLSCATSGFNFNDYFMNWVRQAPGKGLEWIAQIR
NKNYNYATYFAESLEGRFTISRDDSKSSVYLQVNSLRAEDTALYYCTELRYD
YWGQGVMVTVSS
Light:
(SEQ ID NO: 11)

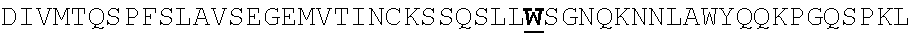

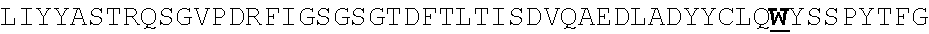

AGTKLEIK

5. L:S31M: The performance is best with the least number of mutations.

Heavy:
(SEQ ID NO: 10)
EVQILETGGGLVKPGGSLRLSCATSGFNFNDYFMNWVRQAPGKGLEWIAQIR
NKNYNYATYFAESLEGRFTISRDDSKSSVYLQVNSLRAEDTALYYCTELRYD
YWGQGVMVTVSS
Light:
(SEQ ID NO: 12)

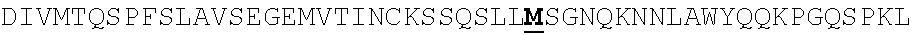

LIYYASTRQSGVPDRFIGSGSGTDFTLTISDVQAEDLADYYCLQHYSSPYTFG
AGTKLEIK

Based on preliminary investigations, Mutants 4 and 5 were selected for further study.

### Example 3: Antibody Production

### Summary:

Variable domains for the new Oslo-2 mAb variant sequences (Mutants 4 and 5) were designed and optimized for expression in mammalian cells (HEK293) prior to being synthesized. The sequences were then subcloned into a cloning and expression vector for the appropriate isotype and subtype of immunoglobulin heavy and light chains (IgG1). HEK293 cells were passaged to the optimum stage for transient transfection. Cells were transiently transfected with heavy and light chain expression vectors and cultured for a further 6-14 days. An 80 ml volume of cells was transfected to obtain approximately 0.5-1.0 mg of antibody. Cultures were harvested and a one-step purification performed using affinity chromatography. Antibody concentration was determined by UV spectroscopy. Antibody yield was determined for all antibodies and aggregation levels were measured by SEC-HPLC.

### Methods:

### Gene Synthesis and Cloning

Variable heavy and variable light domains were designed with appropriate restriction sites at the 5′ and 3′ ends to enable cloning into Absolute Antibody cloning and expression vectors. Variable domains sequences were codon optimized for expression in human cells. Following gene synthesis, the variable domains were cloned into Absolute Antibody vectors of the appropriate species and type. The correct sequence was verified by Sanger sequencing with raw data analyzed using DNASTAR Lasergene software. Once confirmed plasmid DNA preps of the appropriate size were performed to generate a sufficient quantity of high-quality DNA for transfection.

### Expression and Purification

HEK 293 (human embryonic kidney 293) mammalian cells were passaged to the optimum stage for transient transfection. Cells were transiently transfected with heavy and light chain expression vectors and cultured for a further 6 days. Cultures were harvested by centrifugation at 4000 rpm and filtered through a 0.22 μM filter. The first step of purification was performed by Protein A affinity chromatography with elution using citrate pH3.0 buffer followed by neutralization with 0.5M Tris, pH 9.0. Eluted protein was then buffer exchanged into PBS using a desalting column. Antibody concentration was determined by UV spectroscopy and the antibodies were concentrated as necessary.

### Antibody Analytics

Antibody purity was determined by SDS-PAGE (sodium dodecyl sulfate-polyacrylamide gel electrophoresis) and HPLC (high-performance liquid chromatography). SEC-HPLC was performed on an Agilent 1100 series instrument using an appropriate size exclusion column (SEC). Antibody expression titer was determined by Protein A HPLC.

### Results:

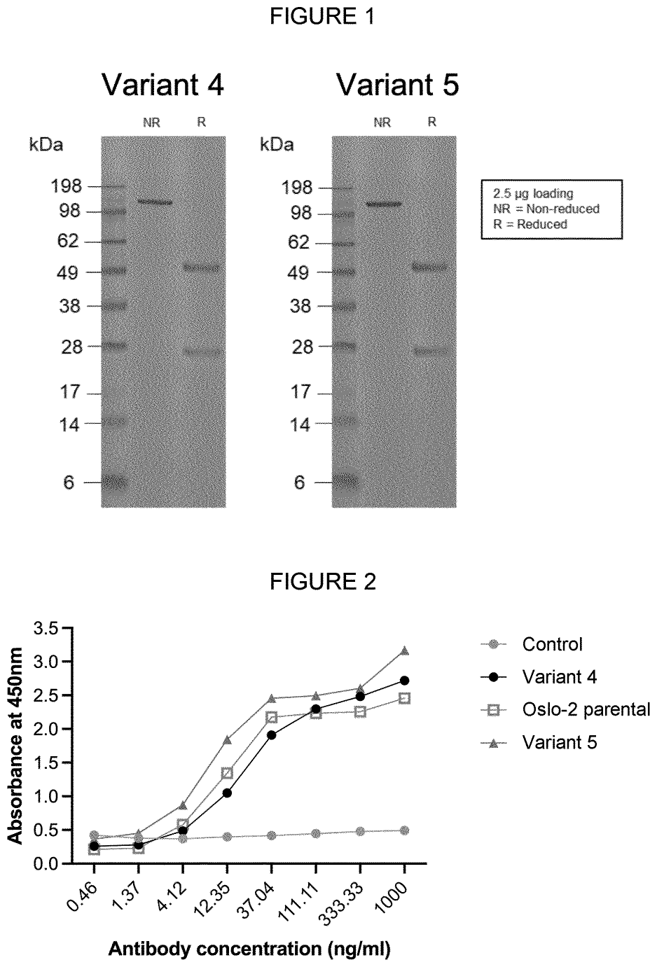

FIG. 1 shows the results of quality control of expression of the new antibodies identified as Mutants 4 and 5 (also referred to as Variants 4 and 5). Cultures from an 80 ml volume of transfected cells were harvested and purification was performed through one-step affinity chromatography. For quality control, the purified antibodies ran on SDS-PAGE gel.

### Example 3: Evaluation of New Antibody Variants Binding Capacity Using Enzyme-Linked Immunosorbent Assay (ELISA)

### Methods:

### Indirect ELISA

Antibody binding was determined by indirect ELISA. A microtitre plate was coated with 50 μL antigen (protein sequence: MPIWKFPDEEGACQPCPINCTHSCVDLDDKGCPAEQRASPLTHHHHHH) (SEQ ID NO: 36) at 2.5 μg/mL for 1 hour with shaking at 300 rpm then blocked with 200 μL 1% casein in PBS for 16 hours at 4° C. The plate was washed twice with PBS containing 0.05% Tween-20. In a separate offline microplate, antibody test samples were prepared in duplicate in a 3-fold serial dilution starting at 3.0 ug/mL. 50 μL of each test sample was transferred to the assay plate and incubated for 1 hour with shaking at 300 rpm. The plate was washed four times with PBS containing 0.05% Tween-20. 50 μL of horse radish peroxidase (HRP) labelled anti-human IgG antibody (catalogue number A8792, Sigma) at a dilution of 1:4000 was added to each well and incubated for 1 hour with shaking at 300 rpm. The plate was washed four times with PBS containing 0.05% Tween-20 and twice with water. 100 μL Tetramethylbenzidine (TMB) substrate (catalogue number 10076433, Fisher) was added and incubated for 10 minutes. The reaction was stopped by addition of 50 μL IM HCl and absorbance was measured at 450 nm using a Byonoy plate reader.

### Results:

Both variants 4 and 5 were shown to be capable of binding to antigen, and furthermore, the absorbance reading of variants 4 and 5 were higher than that of the parental Oslo-2 monoclonal antibody (FIG. 2).

1. A binding molecule which specifically binds p95HER2 comprising the amino acid sequence set forth in SEQ ID NO: 1,
comprising a light chain variable domain (VL) and a heavy chain variable domain (VH) which together form an antigen binding unit, wherein:
(i) the VL comprises three complementarity determining regions (CDRs): LCDR1, LCDR2 and LCDR3, which respectively comprise the amino acid sequences SEQ ID NOs: 2, 3 and 4; and
the VH comprises three CDRs; HCDR1, HCDR2 and HCDR3, which respectively comprise the amino acid sequences SEQ ID NOs: 5, 6 and 7; or
(ii) the VL comprises three complementarity determining regions (CDRs): LCDR1, LCDR2 and LCDR3, which respectively comprise the amino acid sequences SEQ ID NOs: 8, 3 and 9; and
the VH comprises three CDRs; HCDR1, HCDR2 and HCDR3, which respectively comprise the amino acid sequences SEQ ID NOs: 5, 6 and 7.

2. The binding molecule according to claim 1, wherein:
(i) the VH comprises the amino acid sequence set forth in SEQ ID NO: 10, or a sequence with at least 90% identity thereto, and
the VL comprises the amino acid sequence set forth in SEQ ID NO: 11, or a sequence with at least 90% identity thereto; or
(ii) the VH comprises the amino acid sequence set forth in SEQ ID NO: 10, or a sequence with at least 90% identity thereto, and
the VL comprises the amino acid sequence set forth in SEQ ID NO: 12, or a sequence with at least 90% identity thereto.

3. The binding molecule according to claim 1 or 2, wherein the molecule is an antibody or fragment thereof.

4. The binding molecule according to claim 1 or 2, wherein the antigen binding unit is a scFv.

5. The binding molecule according to any one of claims 1 to 4, wherein the molecule is in the form of a conjugate with a cytotoxic agent.

6. The binding molecule according to any one of claims 1 to 4, wherein the molecule is a bi- or tri-specific antibody.

7. The binding molecule according to claim 6, wherein the molecule is a Bi-specific T-cell Engager (BiTE).

8. A Chimeric Antigen Receptor (CAR) comprising an antigen binding unit as defined in any one of claims 1, 2 or 4.

9. The CAR according to claim 7, comprising a human CD8α hinge of SEQ ID NO: 26 or a sequence with at least 90% identity thereto.

10. The CAR according to claim 9, comprising from N-terminal to C-terminal, a human CD8α hinge, a human CD8α transmembrane domain, a human 4-1BB costimulatory domain and a human CD3ζ signaling domain.

11. A nucleic acid encoding the binding molecule according to any one of claims 1 to 7 or the CAR according to any one of claims 8 to 10.

12. A vector comprising the nucleic acid of claim 11.

13. A cytotoxic immune cell expressing a CAR according to any one of claims 8 to 10 in its cell membrane.

14. The cytotoxic immune cell according to claim 13, wherein the cell is a cytotoxic T cell or an NK cell.

15. A pharmaceutical composition comprising a binding molecule according to any one of claims 1 to 7.

16. A pharmaceutical composition comprising a nucleic acid according to claim 11 or a vector according to claim 12.

17. A pharmaceutical composition comprising a cytotoxic immune cell according to claim 13 or 14.

18. A method of treatment of cancer in a human patient comprising the step of administering a binding molecule according to any one of claims 1 to 7, a cytotoxic immune cell according to claim 13 or 14 or a pharmaceutical composition according to any one of claims 15 to 17.

19. A method of treatment of cancer in a human patient comprising the steps:
a. obtaining a sample comprising cancer cells from the patient;
b. analysing whether the cancer cells express p95HER2 by contacting the cells ex vivo with a binding molecule as defined in any one of claims 1 to 7 further comprising a moiety suitable for detection; and
c. administering a chemotherapy to the patient if the cancer cells are p95HER2 positive.

20. A method of diagnosing cancer comprising the steps
a. obtaining a sample comprising cells from a human patient;
b. analysing whether the cells express p95HER2 by contacting the cells ex vivo with a binding molecule as defined in any one of claims 1 to 7, wherein the protein comprises a moiety suitable for detection; and
c. diagnosing the patient with cancer if the cells express p95HER2.

21. A binding molecule according to any one of claims 1 to 7, a CAR according to any one of claims 8 to 10, a cytotoxic immune cell according to claim 13 or 14 or a pharmaceutical composition according to any one of claims 15 to 17 for use in therapy.

22. A binding molecule according to any one of claims 1 to 7, a CAR according to any one of claims 8 to 10, a cytotoxic immune cell according to claim 13 or 14 or a pharmaceutical composition according to any one of claims 15 to 17 for use in the treatment of cancer, wherein the cancer expresses p95HER2 comprising the amino acid sequence set forth in SEQ ID NO: 1.

23. The binding molecule, CAR, cytotoxic immune cell or pharmaceutical composition for use according to claim 22, wherein the cancer is breast cancer.

24. A method of diagnosing cancer in a subject, the method comprising:
(a) contacting a sample of cells from the subject with a binding molecule as defined in any one of claims 1 to 7, wherein the binding molecule further comprises a detection moiety;
(b) determining whether the cells express p95HER2; and
(c) if the cells express p95HER2, diagnosing the patient with cancer.


SEQUENCE LISTING (38 sequences)

SEQ ID NO: 1 - protein, 9 aa, Homo sapiens
  source 1..9: note = p95HER2 epitope
PIWKFPDEE

SEQ ID NO: 2 - protein, 17 aa, synthetic construct
  source 1..17: note = VL CDR1 Kabat (mutant 4)
KSSQSLLWSGNQKNNLA

SEQ ID NO: 3 - protein, 7 aa, Rattus sp.
  source 1..7: note = parental VL CDR2 Kabat
YASTRQS

SEQ ID NO: 4 - protein, 9 aa, synthetic construct
  source 1..9: note = VL CDR3 Kabat (mutant 4)
LQWYSSPYT

SEQ ID NO: 5 - protein, 5 aa, Rattus sp.
  source 1..5: note = parental VH CDR1 Kabat
DYFMN

SEQ ID NO: 6 - protein, 19 aa, Rattus sp.
  source 1..19: note = parental VH CDR2 Kabat
QIRNKNYNYATYFAESLEG

SEQ ID NO: 7 - protein, 5 aa, Rattus sp.
  source 1..5: note = parental VH CDR3 Kabat
LRYDY

SEQ ID NO: 8 - protein, 17 aa, synthetic construct
  source 1..17: note = VL CDR1 Kabat (mutant 5)
KSSQSLLMSGNQKNNLA

SEQ ID NO: 9 - protein, 9 aa, synthetic construct
  source 1..9: note = VL CDR3 Kabat (mutant 5)
LQHYSSPYT

SEQ ID NO: 10 - protein, 116 aa, Rattus sp.
  source 1..116: note = parental VH
EVQILETGGGLVKPGGSLRLSCATSGFNFNDYFMNWVRQAPGKGLEWIAQIRNKNYNYATYFAESLEGRFTISRDDSKSSVYLQVNSLRAEDTALYYCTELRYDYWGQGVMVTVSS

SEQ ID NO: 11 - protein, 113 aa, synthetic construct
  source 1..113: note = VL (mutant 4)
DIVMTQSPFSLAVSEGEMVTINCKSSQSLLWSGNQKNNLAWYQQKPGQSPKLLIYYASTRQSGVPDRFIGSGSGTDFTLTISDVQAEDLADYYCLQWYSSPYTFGAGTKLEIK

SEQ ID NO: 12 - protein, 113 aa, synthetic construct
  source 1..113: note = VL (mutant 5)
DIVMTQSPFSLAVSEGEMVTINCKSSQSLLMSGNQKNNLAWYQQKPGQSPKLLIYYASTRQSGVPDRFIGSGSGTDFTLTISDVQAEDLADYYCLQHYSSPYTFGAGTKLEIK

SEQ ID NO: 13 - protein, 17 aa, Rattus sp.
  source 1..17: note = parental VL CDR1 Kabat
KSSQSLLSSGNQKNNLA

SEQ ID NO: 14 - protein, 20 aa, synthetic construct
  source 1..20: note = leader peptide
MESQTQALISLLLWVYGTYG

SEQ ID NO: 15 - protein, 113 aa, Rattus sp.
  source 1..113: note = parental VL
DIVMTQSPFSLAVSEGEMVTINCKSSQSLLSSGNQKNNLAWYQQKPGQSPKLLIYYASTRQSGVPDRFIGSGSGTDFTLTISDVQAEDLADYYCLQHYSSPYTFGAGTKLELK

SEQ ID NO: 16 - protein, 17 aa, synthetic construct
  source 1..17: note = VL CDR1 (mutant 4 or 5)
  VARIANT 8: note = X can be W or M
KSSQSLLXSGNQKNNLA

SEQ ID NO: 17 - protein, 9 aa, synthetic construct
  source 1..9: note = VL CDR3 (mutant 4 or 5)
  VARIANT 3: note = X can be H or W
LQXYSSPYT

SEQ ID NO: 18 - protein, 645 aa, Homo sapiens
  source 1..645: note = p95HER2 isoform (611-HER2-CTF)
MPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 19 - protein, 5 aa, synthetic construct
  source 1..5: note = linker
GGGGS

SEQ ID NO: 20 - protein, 15 aa, synthetic construct
  source 1..15: note = linker
GGGGSGGGGSGGGGS

SEQ ID NO: 21 - protein, 20 aa, synthetic construct
  source 1..20: note = linker
GGGGSGGGGSGGGGSGGGGS

SEQ ID NO: 22 - protein, 25 aa, synthetic construct
  source 1..25: note = linker
GGGGSGGGGSGGGGSGGGGSGGGGS

SEQ ID NO: 23 - protein, 20 aa, synthetic construct
  source 1..20: note = linker
GGGGSGGGGSAGGGSGGGGS

SEQ ID NO: 24 - protein, 324 aa, Mus musculus
  source 1..324: note = mouse IgG1 constant region
AKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLMISLTPKVTCVVVDISKDDPEVQFSWFVDDVEVHTAQTKPREEQINSTFRSVSELPILHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 25 - protein, 107 aa, Mus musculus
  source 1..107: note = mouse kappa light chain constant region
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 26 - protein, 45 aa, Homo sapiens
  source 1..45: note = human CD8 alpha hinge
SDPTTTPAPRPPTPAPTIASQPLSLRPEACRPAAGGAVHTRGLDF

SEQ ID NO: 27 - protein, 112 aa, Homo sapiens
  source 1..112: note = human CD3 zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 42 aa, Homo sapiens
  source 1..42: note = human 4 1BB co-stimulatory domain
KRGRKKLLYIFKQPFMRPVQTTQEEDGCSCRFPEEEEGGCEL

SEQ ID NO: 29 - protein, 27 aa, Homo sapiens
  source 1..27: note = human CD8 alpha transmembrane domain
ACDIYIWAPLAGTCGVLLLSLVITLYC

SEQ ID NO: 30 - protein, 5 aa, synthetic construct
  source 1..5: note = VH CDR3 (comprises mutation L107W)
WRDYY

SEQ ID NO: 31 - protein, 17 aa, synthetic construct
  source 1..17: note = VL CDR1 (comprises mutations S31W and N38M)
KSSQSLLWSGNQKNMLA

SEQ ID NO: 32 - protein, 9 aa, synthetic construct
  source 1..9: note = VL CDR3 (comprises mutations Y108D and S109W)
LQHDWSPYT

SEQ ID NO: 33 - protein, 116 aa, synthetic construct
  source 1..116: note = mutant VH comprising L107W
EVQILETGGGLVKPGGSLRLSCATSGFNFNDYFMNWVRQAPGKGLEWIAQIRNKNYNYATYFAESLEGRFTISRDDSKSSVYLQVNSLRAEDTALYYCTEWRYDYWGQGVMVTVSS

SEQ ID NO: 34 - protein, 113 aa, synthetic construct
  source 1..113: note = mutant VL comprising S31W, N38M, Y108D and S109W
DIVMTQSPFSLAVSEGEMVTINCKSSQSLLWSGNQKNMLAWYQQKPGQSPKLLIYYASTRQSGVPDRFIGSGSGTDFTLTISDVQAEDLADYYCLQHDWSPYTFGAGTKLEIK

SEQ ID NO: 35 - protein, 16 aa, synthetic construct
  source 1..16: note = signal peptide
MPLLLLLPLLWAGALA

SEQ ID NO: 36 - protein, 48 aa, synthetic construct
  source 1..48: note = antigen in ELISA
MPIWKFPDEEGACQPCPINCTHSCVDLDDKGCPAEQRASPLTHHHHHH

SEQ ID NO: 37 - protein, 330 aa, Homo sapiens [sequence omitted]

SEQ ID NO: 38 - protein, 107 aa, Homo sapiens [sequence omitted]